(19) United States
(12) Patent Application Publication
(10) Pub. No.: US 2009/0287284 A1
(43) Pub. Date: Nov. 19, 2009

(54) Y-SHAPED ELECTRODES FOR USE WITH ELECTRO-STIMULATION DEVICE

(75) Inventors: BENJAMIN SOONG, PLANO, TX (US); TAMMY JANE LAHUTSKY, PLANO, TX (US); TERRYL EVERETT BIGGS, ADDISON, TX (US)

Correspondence Address: HOWISON & ARNOTT, L.L.P, P.O. BOX 741715, DALLAS, TX, 75374-1715, US

(73) Assignee: AVAZZIA, INC., DALLAS, TX (US)

(21) Appl. No.: 12/466,888

(22) Filed: May 15, 2009

Related U.S. Application Data
(60) Provisional application No. 61/053,876, filed on May 16, 2008.

(51) Int. Cl. A61N 1/04

(52) U.S. Cl. 607/115

(57) ABSTRACT

An electro-stimulation probe includes a handle portion having a first end and a second end and a central axis. A pair of electrodes apply an electrical signal to a body of a patient. A rigid member associated with each of the pair of electrodes hold the electrode in a fixed position with respect to the first end of the handle portion. The rigid member holds the electrode at a fixed angle from the central axis of the handle portion.

### CROSS-REFERENCE TO RELATED APPLICATIONS

This application claims priority from U.S. Provisional Application No. 61/053,876 filed on May 16, 2008, which is incorporated herein by reference.

### TECHNICAL FIELD

The present invention relates to electrodes, and more particularly, to y-shaped electrodes for use with an electro-stimulation device.

### SUMMARY

The present invention, as disclosed and described herein, in on aspect thereof, comprises an electro-stimulation probe. The electro-stimulation probe includes a handle portion having a first end and a second end and a central axis. A pair of electrodes are used for applying an electrical signal to a body of a patient. A rigid member associated with each of the pair of electrodes hold the electrodes in a fixed position with respect to the first end of the handle portion. The rigid member holds the electrode at a fixed angle from the central axis of the handle portion.

### BRIEF DESCRIPTION OF THE DRAWINGS

For a more complete understanding, reference is now made to the following description taken in conjunction with the accompanying Drawings in which:

FIG. 1 is a top view of the y-shaped electrode for use with an electro-stimulation device;

FIG. 2 is an end view of the y-shaped electrode for use with an electro-stimulation device;

FIG. 3 is a side view of the y-shaped electrode for use with an electro-stimulation device;

FIG. 4 is a cross-sectional view illustrating the wires of the y-shaped electrode for use with the electro-stimulation device; and

FIG. 5 illustrates the power connection for the y-shaped electrode.

### DETAILED DESCRIPTION

Referring now to the drawings, wherein like reference numbers are used herein to designate like elements throughout, the various views and embodiments of y-shaped electrodes for use with electro-stimulation device are illustrated and described, and other possible embodiments are described. The figures are not necessarily drawn to scale, and in some instances the drawings have been exaggerated and/or simplified in places for illustrative purposes only. One of ordinary skill in the art will appreciate the many possible applications and variations based on the following examples of possible embodiments.

In the medical market, there are a number of different types of electro-stimulation devices that are utilized for providing electronic stimulation to the body of a patient. Various types of electro-stimulation devices include a TENS (transcutaneous electro nerve stimulator) device, an interferential electro nerve stimulator, a PMS (power muscle stimulator) device, an EMS (electro muscle stimulator) device, a biofeedback electro-stimulator and a SCENAR (self-controlled electro nerve stimulator) device. Each of these devices are used for supplying some type of electro-stimulation to the body of a patient.

Typically, the output from these types of electro-stimulation systems are provided through a lead wire to various types of electro-stimulation electrodes that provide the electric stimulation to the body of the patient. Various types of electrodes via which the electronic stimulation is provided include self-adhesive conductive pads which stick to the body of a patient using adhesive tape. Pencil shaped electrodes are shaped like a pencil or pen and can also be used for electro-stimulation. Other types of electrodes comprise y-shaped electrodes. Y-shaped electrodes normally come in two different configurations including a bendable configuration wherein the electrode portion that is applied to the body of a patient connects to the body of the electrode via a flexible member that enables the body contact electrode to be positioned in a variety of locations. Another type of y-shaped electrode comprises electrodes that are rigid and straight extending in the same plane as the handle of the device. The electrodes applied to the body are connected to the body of the electrode via a straight and rigid member. Finally, vaginal or anal electrodes may be used for applying electro-stimulation to these portions of an individual's body.

Other types of electrode devices include comb electrodes which use a brush or comb-shaped device for providing electro stimulations to hair follicles of an individual. Conductive garments such as a conductive glove, sock, arm sleeve or leg sleeve may be worn upon a person's body, and the electro-stimulation is then provided through the conductive garment to the associated portion of an individual's body. Finally, conductive wraps including a Velcro connection may be used for wrapping around and treating a patient's joints for afflictions such as carpel tunnel syndrome, and may be utilized as arm, leg, back, knee, ankle and elbow wraps. In these devices, the electrodes are embedded within an associated sleeve and the sleeve is then attached to the desired portion of a patient's body via Velcro connection.

Each of these types of electrode application devices suffer from a number of inherent problems. Each of the designs are not suitable for accessing hard to reach places on the back of a patient making self electrotherapy operations difficult to achieve with respect to a patient's back. Additionally, the angle required to keep the electrode handle from scraping the skin tissue of a patient when the ball electrode is on the end of a y-shaped type electrode presents a problem. Additionally, length of the handle can make application of the electrode to various portions of the patient's body somewhat challenging. The bendable y-shaped electrodes described hereinabove will not maintain their shape when they are firmly pressed into the tissue of a patient for a soft tissue application of electrotherapy. The flexible nature of the y-shaped electrodes causes the electrode to deform when any type of firm pressure is applied.

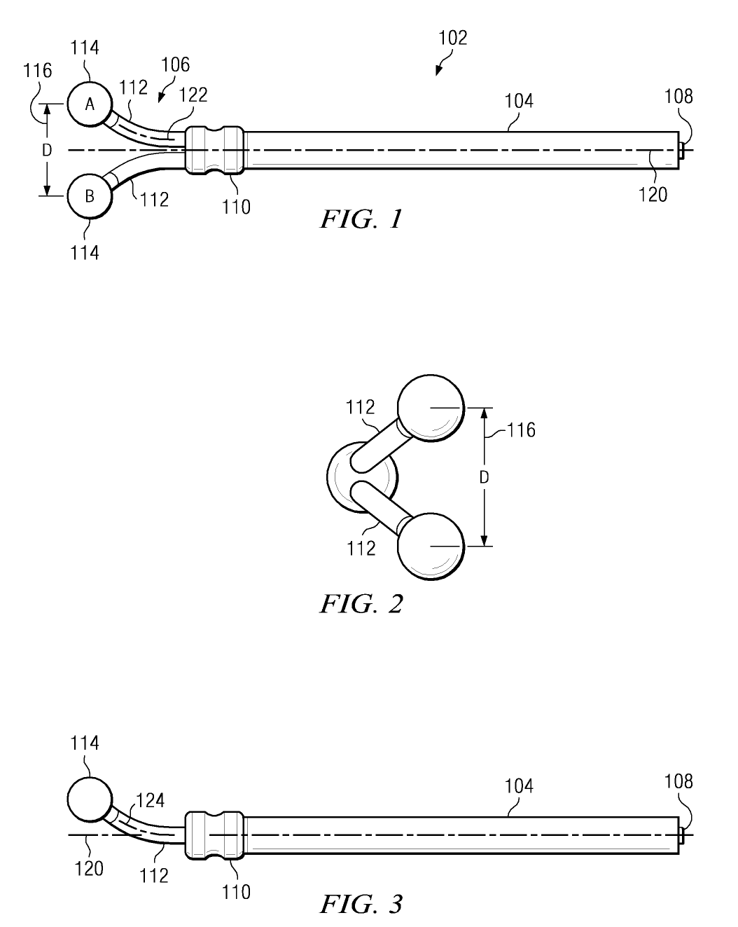

Referring now to the drawings, and more particularly to FIGS. 1-3, there are illustrated a top, end and side view respectively of a y-shaped electrode for use with an electro-stimulation device. The y-shaped electro-stimulation device 102 provides for a handheld electronic probe that provides a probe angle that is useful within an electro-stimulation device. The y-shaped electrode includes a bend or curve off of the y electrode knobs which allow the user of the device to penetrate the electrode knobs into an area of a patient being treated without any undue interference with the patient's body. Referring now more particularly to FIG. 1, there is illustrated a top view of the electro-stimulation electrode 102. The y-shaped electrode 102 includes a body portion 104 connected with an electro stimulation portion 106. The body portion 104 in one embodiment is made of a plastic or injection molded material and acts as the handle of the y-shaped electronic probe 102. A first end of the body portion 104 provides a connector 108 for interconnecting the y-shaped electronic probe 102 with a wire that provides the electro stimulation signal to the electrode. In a preferred embodiment, the connector 108 comprises a stereo connector for accepting a standard stereo jack style input. The body portion 104 has a sufficient length to enable a patient to apply the electro stimulation portion 106 directly to their own back without requiring the services of another individual for electro stimulating the individual's back. In one embodiment, the length of the handle will comprise approximately 185.2 mm in length while the electro stimulation portion will comprise approximately 75.5 mm in length. This will enable an overall sufficient length enabling a user to easily apply the electro-stimulation portion 106 to the back of a user.

The electro-stimulation portion 106 includes a connecting collar 110 which enables the electro-stimulation portion 106 to be interconnected with the body portion 104. The electro-stimulation portion 106 comprises and interchangeable portion that may be removed from the body portion 104 to enable the connection of another electro-stimulation portion to the body portion. In this manner, multiple types of electro-stimulation portions 106 of various sizes may be used with the device. The connecting collar 110 may connect the electro-stimulation portion 106 with the body portion 104 via a threaded connection, snap-on connection or other means for interconnecting the portions in a fixed relationship. Extending from the collar portion 110 are a pair of rigid curved members 112. The rigid curved members 112 curve upward from the connecting collar 110 to the electrode spheres 1 14. The electrode spheres 114 may be of varying sizes to accommodate various treatment areas and techniques. The electrode spheres 114 and rigid curved members 112 enable application of the electrode spheres 114 to the body of a patient. The rigid nature of the rigid curved members 112 enable a force to be applied by the handle to the spheres 114 such that the electrode spheres 114 may be applied to soft tissue areas of a patient with a great deal of pressure. The rigid curved members 112 also hold the electrode spheres 114 at a fixed angle from the central axis of the body portion 104. The rigid curved members 114 also hold the electrode spheres at a fixed distance from each other. The distance between the electrode spheres 114 may be varied for different electro-stimulation portions to cover different treatment areas and techniques. The rigid curved members 112 are curved both in the horizontal plane at a certain angle from a center line 120 of the y-shaped probe 102 to provide an arc 122 in the horizontal plane as illustrated generally in FIG. 1. Additionally, the rigid curve member 112 is curved in the vertical plane at an angle from the center line 120 in a vertical direction to provide a vertical curve 124 as illustrated in FIG. 3. Each of these curves provides an ability for providing the electrode spheres 114 at a fixed distance from the body portion 104 of the y-shaped electrode 102. The electrode spheres 114 and curved rigid members 112 may be made of a medical grade metal such as surgical steel.

The electrode spheres 114 are of a size necessary for effective and comfortable soft tissue therapy. The distance D 116 between the electrode spheres 114 may vary depending upon the particular application of the y-shaped probe. For application to the gum tissue of a patient, the center to center distance D 116 of the spheres may be approximately 12.7 mm. For application of the electrode to larger muscles of an individual such as their back, thigh, calf or arm muscle regions the center to center distance D 116 of the electrode spheres 114 may be from 31.0-45.0 mm. Finally, for application to the spinal region of an individual up and down the spinal column, a center to center distance of 57.0-70.0 mm may be used. This larger width application would be beneficial for therapy for conditions such as curvature of the spine. In these various applications, the rigidity, size and spacing of the balls may all be adjusted depending upon the particular application of the device.

As can be seen in FIG. 2, the handle portion 104 may comprise a cylindrical member which enables a user to easily grasp the body portion 104 for use as a handle. In an alternative embodiment, the handle portion 104 rather than being a cylinder may be configured using an ergonomic design that shapes the body portion 104 to easily fit the hand of a user and include indentions therein for the fingers of the user or include a curved shape enabling better grasping by the hand of a user.

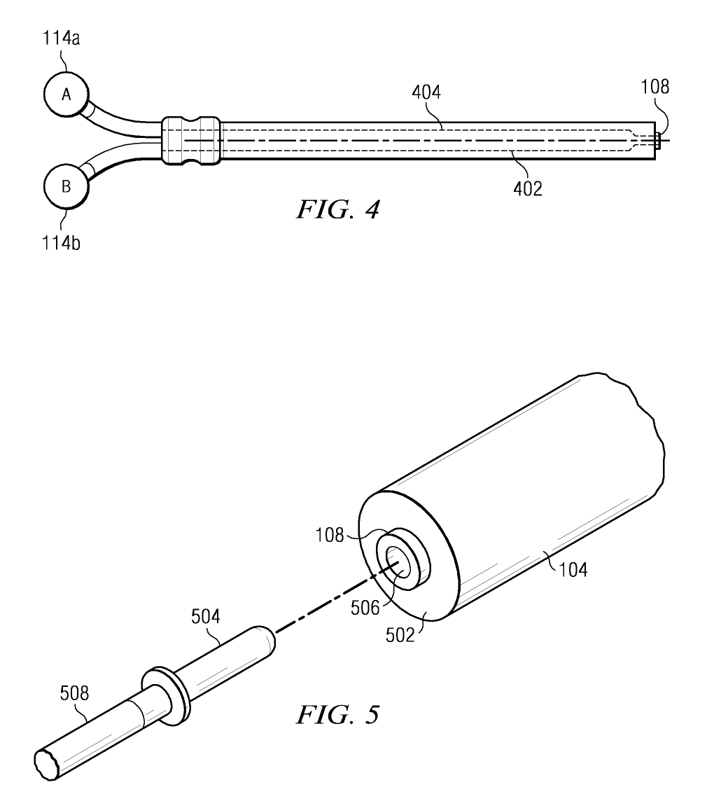

Referring now to FIG. 4, there is illustrated a cutaway view of the top view of the y-shaped electrode 102. This illustrates the pair of wires 402 and 404 that run from the connector 108 to each of the electrode spheres 114. The wire 402 provides an interconnection between the connector 108 and the spherical electrode 114b that is applied to a patient's body. Similarly, the wire 404 provides an interconnection between the electrical connector 108 and the spherical electrode 114a. While the preferred embodiment illustrated in FIG. 4 illustrates providing a connection between the lead wires 402 and 404 via an electrical plug connection 108, in alternative embodiments, a lead wire may provide a continuous connection from the wires 402 and 404 to the y electrode such that a removable plug is not provided. Further, a direct wire connection would be provided. The preferred embodiment utilizes a connector 108 that comprises a 2.5 mm stereo jack to provide connections to spherical electrode 114a and spherical electrode 114b. A further connection is reserved for a ground connection that is not connected to either spherical electrode 114.

The use of a plug feature in connector 108 provides a significant improvement over prior art implementations because it allows for the lead wires 402 and 404 to be replaced without affecting the life of the electrode 102. Use of the plug connection for the connector 108 limits the possibility of a worn or broken lead wire that wears out with continued use and provides strain relief for the wire connection with the y-shaped electrode 102. Referring now to FIG. 5, there is provided a further illustration of the stereo plug electrical connector 108. The plug connector 108 is located on the base 502 of the body portion 104. The connector 108 is configured to receive a stereo plug connector 504 that inserts within a hole 506 of the connector 108. The stereo connector 504 provides an ease of connection with a wire 508 that provides the electro stimulation signal to the lead wires 402 and 404 described with respect to FIG. 4.

The above-described configuration will provide a number of advantages over prior art electro-stimulation probes. The rigid and curved nature of the members of the probe enable the electrode spheres to be pressed and rubbed into the skin tissue of a patient without the handle scraping the skin of the patient due to the curve shaped of the probe arms sufficiently removing the body portion 104 of the y-shaped probe from the electrode spheres 114. Additionally, the electrode balls are able to protrude out from the body portion at an angle instead of straight within the same plane enabling better application of the spherical electrodes to the back of a patient and other hard to reach areas. Protrusion outward from the body portion 104 has been reduced significantly to avoid excess handle width that might interfere with the application of the probes to a body of a patient. The electrical connection is made via a single plug to the unit enabling the lead wires to be changed and replaced or provide strain relief to the lead wire.

It will be appreciated by those skilled in the art having the benefit of this disclosure that this y-shaped electrodes for use with electro-stimulation device provides an improved configuration of the y-shaped electrodes. It should be understood that the drawings and detailed description herein are to be regarded in an illustrative rather than a restrictive manner, and are not intended to be limiting to the particular forms and examples disclosed. On the contrary, included are any further modifications, changes, rearrangements, substitutions, alternatives, design choices, and embodiments apparent to those of ordinary skill in the art, without departing from the spirit and scope hereof, as defined by the following claims. Thus, it is intended that the following claims be interpreted to embrace all such further modifications, changes, rearrangements, substitutions, alternatives, design choices, and embodiments.

What is claimed is:

1. An electro-stimulation probe, comprising:
a handle portion having a first end and a second end and a central axis;
a pair of electrodes for applying an electrical signal to a body of a patient; and
a rigid member associated with each of the pair of electrodes for holding the electrodes in a fixed position with respect to the first end of the handle portion, wherein the rigid member holds the electrode at a fixed angle from the central axis of the handle portion.

2. The electro-stimulation probe of claim 1, wherein the rigid member defines a curve in at least one direction.

3. The electro-stimulation probe of claim 1, further including an electrical connector located at the second end of the handle portion, the electrical providing the electrical signal to the electro-stimulation probe.

4. The electro-stimulation probe of claim 3, wherein the electrical connector further comprises a stereo connector.

5. The electro-stimulation probe of claim 3, further including:
a first wire for providing a connection between the connector and a first electrode of the pair of electrodes; and
a second wire for providing a connection between the connector and a second electrode of the pair of electrodes.

6. The electro-stimulation probe of claim 5, wherein the first wire and the second wire are removable.

7. The electro-stimulation probe of claim 1, wherein the pair of electrodes comprise spherical electrodes.

8. The electro-stimulation device probe of claim 1, wherein the handle portion has sufficient length to enable a user to apply the pair of electrodes to a back of the user.

9. An electro-stimulation probe, comprising:
a handle portion having a first end and a second end and a central axis;
a pair of spherical electrodes for applying an electrical signal to a body of a patient;
a rigid member associated with each of the pair of electrodes for holding the electrode in a fixed position with respect to the first end of the handle portion, wherein the rigid member holds the electrode at a fixed angle from the central axis of the handle portion; and
an electrical connector located at the second end of the handle portion, the electrical providing the electrical signal to the electro-stimulation probe.

10. The electro-stimulation probe of claim 9, wherein the rigid member defines a curve in at least one direction.

11. The electro-stimulation probe of claim 9, wherein the electrical connector further comprises a stereo connector.

12. The electro-stimulation probe of claim 9, further including:
a first wire for providing a connection between the connector and a first electrode of the pair of electrodes; and
a second wire for providing a connection between the connector and a second electrode of the pair of electrodes.

13. The electro-stimulation probe of claim 12, wherein the first wire and the second wire are removable.

14. The electro-stimulation device probe of claim 9, wherein the handle portion has sufficient length to enable a user to apply the pair of electrodes to a back of the user.

15. An electro-stimulation probe, comprising:
a handle portion having a first end and a second end and a central axis;
a pair of electrodes for applying an electrical signal to a body of a patient;
a rigid member associated with each of the pair of electrodes for holding the electrode in a fixed position with respect to the first end of the handle portion, wherein the rigid member holds the electrode at a fixed angle from the central axis of the handle portion, wherein the rigid member further defines a curve in at least one direction; and
an electrical connector located at the second end of the handle portion, the electrical providing the electrical signal to the electro-stimulation probe.

16. The electro-stimulation probe of claim 15, wherein the electrical connector further comprises a stereo connector.

17. The electro-stimulation probe of claim 15, further including:
a first wire for providing a connection between the connector and a first electrode of the pair of electrodes; and
a second wire for providing a connection between the connector and a second electrode of the pair of electrodes.

18. The electro-stimulation probe of claim 17, wherein the first wire and the second wire are removable.

19. The electro-stimulation probe of claim 15, wherein the pair of electrodes comprise spherical electrodes.

20. The electro-stimulation device probe of claim 15, wherein the handle portion has sufficient length to enable a user to apply the pair of electrodes to a back of the user.
